(19) United States
(12) Patent Application Publication
(10) Pub. No.: US 2019/0032086 A1
(43) Pub. Date: Jan. 31, 2019

(54) METHOD FOR PREPARING A GENE KNOCK-OUT CANINE WITH SOMATIC CELL CLONING TECHNOLOGY

(71) Applicant: Beijing Sinogene Biotechnology Co. Ltd., Beijing (CN)

(72) Inventors: Min Zheng, Beijing (CN); Jianping Zhao, Beijing (CN); Jidong Mi, Beijing (CN)

(21) Appl. No.: 16/045,683

(22) Filed: Jul. 25, 2018

(30) Foreign Application Priority Data
Jul. 25, 2017 (CN) 201710614324.8

(51) Int. Cl. C12N 15/877; A01K 67/027

(52) U.S. Cl. CPC C12N 15/877; A01K 67/0273; A01K 2267/03; A01K 2227/10; A01K 2217/075

(57) ABSTRACT

The present invention relates to a method for preparing a gene knock-out canine with use of somatic cell cloning technology, in particular relates to a method for preparing a gene knock-out canine with use of somatic cell cloning technology using a fusion liquid of low osmotic pressure together with autologous embryo transplanting.

### CROSS-REFERENCE TO RELATED APPLICATION

This non-provisional application claims priority to and benefit of, under 35 U.S.C. § 119(a), Patent Application No. 201710614324.8 filed in P.R. China on Jul. 25, 2017, the entire content of which is hereby incorporated by reference.

### FIELD OF THE INVENTION

The present invention relates to a method for preparing a gene knock-out canine with use of somatic cell cloning technology, more particularly to a method for preparing a gene editing canine with use of somatic cell cloning technology using a fusion liquid of low osmotic pressure together with autologous embryo transplanting.

### BACKGROUND OF THE INVENTION

Canine is one of the commonly used experimental animals in fundamental medical research and teaching, and especially, plays an important role in experimental researches such as physiology, pharmacology and pathophysiology. Through complete genomic sequencing and analysis, totally 19,300 genes of canines have been determined, wherein about 18,000 genes are the same as known human genes, the similarity of canines' genome to that of human is higher than mice and other experimental animals. Canine is also very similar to human in terms of hereditary diseases, and more than 360 hereditary diseases including cancer, cardiac disease, deaf-mute, blindness and immune nervous system diseases are the same as those for human, and is proper for being used as a model animal in study of human diseases. Further, canine has less hereditary diseases, good experimental repeatability, well-developed blood circulation and nervous system. Canine is similar in digestive system and internal organs, and is closer in toxicological reactions to that of human. Therefore, canine is especially suitable for researches in pharmacology, circulatory physiology, ophthalmology, toxicology, and surgery, etc. Further, canine is gentle and easy to tune, and can cooperate well in experimental research through short-term training. Therefore, canine is regarded to be comparatively ideal for experimental research in international medical and biological fields.

Currently used methods for establishing a canine disease model mainly include the methods, such as feeding method, mechanical damage method, immunization method, etc. Feeding method, mechanical damage method and immunization method are to use special ways to induce healthy animals to have disease phenotypes. These methods have problems that the inductive canine animal models do not show disease phenotypes, the duration of phenotype thereof is short, or the inductive canine animal models cannot simulate human disease symptoms. Using a gene engineering method for gene knock-out or transgenic modification of canine genome, the disease symptoms are primary symptoms, the disease phenotype can last a long period of time, and the disease is hereditary.

An animal somatic cell cloning technology transfers cells into receptor oocyte through nucleus transplanting to produce individual animals having the same DNA sequence information as that of the donor cell. The most important feature of the technology is that the born cloning animals have completely consistent genetic information to that of the donor cell. Therefore using cloning technology can duplicate animal somatic cell; cloning technology can be used for the production of transgenic animals, regeneration of excellent livestocks, preservation of resources of endangered animal resources, therapeutic cloning, etc.

However, canine has a greatly different reproductive physiology to that of other mammal animals, which causes extreme difficulties when in vitro operation of canine oocyte and embryo, the establishment of a gene knock-out or a transgenic modification canine and a somatic cell cloning canine are usually considered to be difficult to succeed.

In general, there are problems in the technology for preparing a gene knock-out canine in prior art: (1) normally fertilized embryo of canine is generally used for cytoplasmic injection, the weakness of the technology is that as the canine fertilized embryo is under a development period, the injected fertilized embryo may cause inconsistent in types of gene knock-out of different blastomeres during the cleavage process, i.e., a chimera of gene knock-out canine may be obtained; (2) gene knockout canines have not yet been obtained using a gene targeting technique, and stable knockout cloned canines having a knockout efficiency of 100% have not been obtained through somatic cell cloning of the gene knock-out canines; and (3) female canines of synchronous oestrus are normally selected for allogenic embryo transplanting, which may have low successful rate, and increase cost of cloning canine, further, allogenic embryo transplanting requires a large amount of oestrous female canines and complex operating means, which leads to difficulties in achieving a industrial production of cloning canines.

Therefore, there is a need in developing an effective method to improve canine cloning efficiency, and also a method to obtain a somatic cell cloning canine which reserves all gene knock-out characters of a gene knock-out canine that cannot be obtained easily.

### SUMMARY OF THE INVENTION

The present invention carries out somatic cell cloning for a gene knock-out canine prepared through fertilized embryo cytoplasmic injection, obtains gene knock-out somatic cell cloning canine, uses fusion liquid having a low osmotic pressure, and improves embryo's fusion efficiency; further, oocyte is taken from an embryo transplanting receptor canine of which only one side of the fallopian tubes is flushed, and is transplanted in another the fallopian tube being not embryo flushed side together with cloning embryo obtained from other donor canines, which achieves autologous/allogenic combined embryo transplanting and improves pregnant efficiency of cloning canine. Furthermore, the present invention may carry out cloning by using any identified to be gene knock-out somatic cells, i.e., any somatic cells, of which the target gene is completely silenced, and obtain gene knock-out canines having up to 100% gene knock-out efficiency, a stable gene knock-out effect without having any chimera.

At the first aspect, the present invention provides a somatic cell cloning method of a gene knock-out canine, the method comprises the following steps: (1) preparing a targeting gene knock-out somatic cell as a nuclear donor; (2) preparing an enucleated oocyte from a receptor female canine; (3) introducing a gene knock-out somatic cell into cytoplasm of the enucleated oocyte and constructing a cloned embryo; (4) activating the cloned embryo; and (5) transplanting the cloned embryo obtained in step (4) to the receptor female canine; wherein the enucleated oocyte is obtained from the embryo flushed fallopian tube of a receptor female canine of which only one of fallopian tubes has been embryo flushed, and in step (5), the cloned embryo is transplanted into the fallopian tube of the receptor female canine, which is not embryo flushed.

In preparing an enucleated oocyte from a receptor female canine in step (2), firstly, it needs to identify a female canine that has entered the oestrous period. Specifically, detecting vagina smear, blood drawing to detect the content of progesterone, ooplasmin and luteinizing hormone need to be done every day, when a keratinocyte ratio is above 80-90% and a progesterone level is up to about 4-7 ng/mL, it is identified that the female canine is under ovulatory period. After 72 h-120 h of ovulation, single side of the fallopian tubes is embryo flushed for obtaining a matured oocyte, and then the matured oocyte is subjected to enucleating.

The gene knock-out somatic cell can be a somatic cell of a gene knock-out canine obtained by knocking-out an endogenous gene of a canine oosperm with the use of gene knock-out technology, wherein the somatic cell is a gene knock-out somatic cell whose targeting gene is completely silenced via identification.

The gene knock-out somatic cell can be a gene knock-out somatic cell which is obtained by knocking out an endogenous gene of a canine somatic cell with gene knock-out technology, and selecting those whose targeting gene is completely silenced via identification.

The gene knock-out somatic cell can be a somatic cell of a gene knock-out canine obtained by knocking out an endogenous gene of a canine fertilized ovum with gene knock-out technology, wherein the gene knock-out canine can be a chimera, but the gene knock-out somatic cell is that whose targeting gene is completely silenced via identification.

Preferably, the gene knock-out technology of the targeting gene knock-out can be clustered regularly interspaced short palindromic repeat sequences technology (CRISPR/Cas9), Zinc finger nuclease technology (ZFN), transcriptional activator effector nuclease technology (TALENs) and homologous recombination technology, or any similar gene knock-out technique.

Preferably, in the above step (3), the gene knock-out somatic cell is introduced into the cytoplasm of the enucleated oocyte through electrofusion with use of fusion liquid having an osmotic pressure of 200 mOSM-280 mOSM.

Preferably, in above step (3), the gene knock-out somatic cell is introduced into cytoplasm of the enucleated oocyte through electrofusion with use of a fusion liquid having an osmotic pressure of 240 mOSM.

The fusion liquid is composed of 0.2-0.28M mannitol, 0.1 mM MgSO4, 0.5 mM Hepes and 0.05% BSA.

Preferably, the fusion liquid is composed of 0.24M mannitol, 0.1 mM MgSO4, 0.5 mM Hepes and 0.05% BSA.

Preferably, the electrofusion adopts a voltage of 2-4 kv/cm.

The gene knock-out somatic cell can also be introduced into cytoplasm of the enucleated oocyte through intracytoplasmic injection; the intracytoplasmic injection generally uses a Piezo microinjection system to directly inject the entire donor cell or donor cell nucleus into the cytoplasm. The method of intracytoplasmic injection does not need to carry out electrofusion.

At the second aspect, the present invention provides a somatic cell cloning method of APOE gene knock-out canine, the method comprises the following steps: (1) preparing an APOE gene knock-out somatic cell as a nuclear donor; (2) preparing an enucleated oocyte from a receptor female canine; (3) introducing a gene knock-out somatic cell into cytoplasm of the enucleated oocyte and constructing a reconstructed embryo; (4) activating the cloned embryo; (5) transplanting the cloned embryo obtained in step (4) into the receptor female canine;

wherein the APOE gene Exon3 of the APOE gene knock-out somatic cell comprises the following mutated sequence:

(SEQ ID NO: 1)
cctggaccagggaggct;

the enucleated oocyte is obtained from the embryo flushed fallopian tube of a receptor female canine of which only one of fallopian tubes has been embryo flushed; and

in the above step (5), the cloned embryo is transplanted into the fallopian tube of the receptor female canine, which is not embryo flushed.

Preferably, the APOE gene Exon3 of the APOE gene knock-out somatic cell comprises the following mutated sequence:

(SEQ ID NO: 2)
ctggagcgcgagctggagccgaaggtccagcaggagccctggaccaggga
ggctctgggaggc.

Preferably, the APOE gene Exon3 of the APOE gene knock-out somatic cell has the following sequence:

(SEQ ID NO: 3)
gatgctgggccgatgtgcagccggagccggagctggagcgcgagctggag
ccgaaggtccagcaggagccctggaccagggaggctctgggaggeggcgc
tggcccgcttctgggattacctgcgctgggtgcagacgctgtctgaccag
gtgcaagagggcgtgctcaacacccaggtcacccaggaactgac.

In preparing enucleated oocyte from receptor female canine in step (2), firstly, it needs to identify a female canine that has entered the oestrous period. Specifically, detecting vagina smear, blood drawing to detect the content of progesterone, ooplasmin and luteinizing hormone need to be done every day, when a keratinocyte ratio is above 80-90% and a progesterone level is up to about 4-7 ng/mL, it is identified that the female canine is under ovulatory period. After 72 h-120 h of ovulation, single side of the fallopian tubes is embryo flushed for obtaining a matured oocyte, and then the matured oocyte is subjected to enucleating.

Preferably, the APOE gene knock-out somatic cell is Ear fibroblast BGD-APOEKO-EF0 of APOE gene knock-out beagle canine, which is deposited in China General Microbiological Culture Collection Center (CGMCC) on Mar. 1, 2017 with a CGMCC deposit No. 13804.

The gene knock-out somatic cell can be a somatic cell of a gene knock-out canine obtained by knocking out an endogenous gene of a canine fertilized ovum with gene knock-out technology, wherein the somatic cell is a gene knock-out somatic cell whose targeting gene is completely silenced via identification.

The gene knock-out somatic cell can be a gene knock-out somatic cell which is obtained by knocking out an endogenous gene of a canine somatic cell with gene knock-out technology, and selecting those whose targeting gene is completely silenced via identification.

The gene knock-out somatic cell can be a somatic cell of a gene knock-out canine obtained by knocking out an endogenous gene of a canine fertilized ovum with gene knock-out technology, wherein the gene knock-out canine can be a chimera, but the gene knock-out somatic cell is that whose targeting gene is completely silenced via identification.

The gene knock-out technology of the APOE gene knock-out can be clustered regularly interspaced short palindromic repeat sequences technology (CRISPR/Cas9), Zinc finger nuclease technology (ZFN), transcriptional activator effector nuclease technology (TALENs) and homologous recombination technology, or any similar gene knock-out technique.

Preferably, in the above step (3), the gene knock-out somatic cell is introduced into the cytoplasm of the enucleated oocyte through electrofusion with use of fusion liquid having an osmotic pressure of 200 mOSM-280 mOSM.

Preferably, in the above step (3), the gene knock-out somatic cell is introduced into cytoplasm of the enucleated oocyte through electrofusion with use of a fusion liquid having an osmotic pressure of 240 mOSM.

The fusion liquid is composed of 0.2-0.28M mannitol, 0.1 mM MgSO4, 0.5 mM Hepes and 0.05% BSA.

Preferably, the fusion liquid is composed of 0.24M mannitol, 0.1 mM MgSO4, 0.5 mM Hepes and 0.05% BSA.

Preferably, the electrofusion adopts a voltage of 2-4 kv/cm.

The gene knock-out somatic cell can also be introduced into cytoplasm of the enucleated oocyte through intracytoplasmic injection; the intracytoplasmic injection generally uses a Piezo microinjection system to directly inject the entire donor cell or donor cell nucleus into the cytoplasm. The method of intracytoplasmic injection does not need to carry out electrofusion.

The somatic cell of the present invention can be from various tissues or organs, such as cells from fetal tissue, skin, muscle, ear, breast, fallopian tube, ovary, blood, urine, fat, marrow, blood vessel and endothelium of the lumen. Examples of somatic cells can be used in the present invention include but not limited to fetal fibroblast, skin cell, epithelial cell, ear cell, fibroblast, endothelial cell, muscle cell, breast cell, fallopian tube cell, ovary cell, cumulus cell, nervous cell and osteoblast.

The method of preparing a gene targeting canine in the prior art is generally subjecting a fertilized ovum to gene targeting (gene knock-out or gene embedding). However, such a gene targeting has a low efficiency, the gene mutation type cannot be controlled, and a high occurring rate that the targeting gene is not mutated or only a single-stranded DNA is muted or chimera is occurred in the born baby canines.

The present invention prepares a gene knock-out canine by using somatic cell nucleus transplanting technology, and uses somatic cell identified to be gene knock-out as a nuclear donor, all the born cloned baby canines are gene knock-out baby canines, and the gene knock-out efficiency is up to 100%. Particularly, though identifying the donor cell, the mutating type can be determined, and the somatic cell whose targeting gene is completely silenced is selected as a nuclear donor for cloning, then the gene knock-out effect is stable, and there is no existence of chimera. Comparatively, in the prior art, even the latest technology of injecting mRNA of CRISPR/Cas9 into a fertilized ovum of the canine is used for gene knock-out, the gene knock-out efficiency is still comparatively as low as about 10-20%, the gene mutating type cannot be controlled, and there is a high occurring rate that the targeting gene is not mutated, only a single-stranded DNA is muted or chimera is occurred in the born baby canines.

In the method of the present invention, the receptor female canine itself also provides oocyte, and the oocyte is obtained by embryo flushing only one side of fallopian tubes; and the cloned embryo obtained through nucleus transplanting, electrofusion and activating is transplanted into the fallopian tube which is not embryo flushed of the receptor female canine. Therefore, certainly, the above two technology solutions must involve autologous transplanting, which greatly reduces the amount of experimental canines comparing to requiring a large amount of oestrous female canines in allogenic transplanting in the prior art. In the prior art, normally, more than 40 canines are required, while in the method of the present invention, only several experimental canines are required.

Further, in the prior art, synchronous oestrus female canines are selected for allogenicembryo transplanting. However, it is not easy to identify estrus of female canine; and the accuracy rate of synchronization identification indicated by estrus is low, which also results a low success rate of allogenic embryo transplanting. In contrast, the autologous transplantation necessarily included in the technical solution of the present invention largely avoids the step of judging the synchronization by estrus. Moreover, autologous transplanting can greatly increase implantation efficiency of cloning embryo, thus achieving the purpose of reducing cost and producing cloning canines at a high efficiency. Autologous transplantation can greatly improve the implantation efficiency of cloned embryos, thereby achieving the goal of reducing the production cost of cloned canines and efficiently producing cloned canines.

In addition, the present invention uses a fusion liquid having an osmotic pressure of 200-280 mOSM, which, comparing with a fusion liquid having osmotic pressure of 280-310 mOSM in the prior art, remarkably increases fusion efficiency of embryo.

Abbreviations and definitions of key terms:

Cloning: producing individual animals having the same DNA sequence as that of the donor cell through corresponding technical means.

NT: somatic cell nucleus transplanting, a method to transplant canine somatic cell into enucleated canine oocyte to construct cloned embryo.

DC: donor cell, a cell comprises complete genetic materials, to transplant thereof into receptor oocyte for preparing somatic cell cloned animals.

AET: autologous embryo transplanting, after a MII oocyte is flushed out from oestrous female canine for somatic cell nucleus transplanting, the embryo flushed female canine is as a receptor for cloned embryo transplanting in order to prepare a somatic cell cloned canine.

APOE: an apolipoprotein E, which is one of the apolipoproteins synthesized mainly in liver and brain tissue, and is a constituent of nervous system and plasma lipoproteins. APOE participates in metabolism process of cholesterol and triglyceride in blood by bonding low-density lipoprotein receptor to take in low-density lipoprotein.

ICI: intracytoplasmic injection, which refers to injecting a gene into cytoplasm of fertilized ovum through micromanipulation with use of a microinjection needle.

AS: atherosclerosis. Lipid metabolism disorder is the lesion foundation of atherosclerosis. The disease is characterized in that lesion of the involved arterial starts from endometrium. Accumulation of lipid and complex carbohydrate occurs at first in general, followed by bleeding and thrombosis. Hereafter, proliferation and calcinosis of fibrous tissue are further developed, and gradual metamorphosis and calcification of arterial media happen, which lead to thickening and hardening of arterial wall and vascular stenosis. The lesion often involves large and medium muscular artery. Once the lesion is developed enough to block the artery cavity, the tissue or organ supplied by the arteries will be ischemic or necrotic. Since the lipid accumulated in the arterial intimas is yellow atherosclerosis, it is called atherosclerosis.

### BRIEF DESCRIPTION OF THE DRAWINGS

FIG. 1 shows a sequence alignment of the characteristic sequence of APOE gene from ear tissue and tail tissue of two gene knock-out canines numbered 161207 and 170111 compared with wild type canine.

FIG. 2 shows a comparison of gene sequence peaks of ear tissue and tail tissue of the APOE gene knock-out canine-Apple numbered 161207 to those of the wild type canine numbered 161206.

FIG. 3 shows a sequence alignment of the APOE gene knock-out canine, i.e., the somatic cell donor canine, numbered 161207 to APOE gene knock-out cloning canines numbered 170502 (Long Long), 170610 and 170611, respectively.

FIG. 4 shows a comparison photograph of the APOE gene knock-out canine-Apple (FIG. 4A) numbered 161207 to the cloned canine “Long Long” (FIG. 4B) numbered 170502, 30 days after birth.

### DETAILED DESCRIPTION OF THE INVENTION

Technical solutions of the present invention are further described in below through combining examples and drawings of the Description. These examples are for illustrating rather than setting limit to the scopes of protection of the present invention.

### EXAMPLES

(1) Preparing an APOE Gene Knock-Out Canine

Preparing an APOE gene knock-out canine comprises the following steps:

(1) determining a targeting site sequence directed to a sequence of an exon based on the canine APOE gene sequence;

(2) synthesizing sgRNA sequence and the complementary sequence thereof according to the targeting site sequence determined in step (1), then the synthesized sequence is connected to skeleton vector to construct a sgRNA targeting vector;

(3) in vitro transcription of sgRNA targeting vector to obtain mRNA of sgRNA, and in vitro transcription of CRISPR/Cas9 to obtain mRNA;

(4) mixing mRNA of sgRNA and mRNA of CRISPR/Cas9 obtained in step (3) and then intracytoplasmic injection thereof into a fertilized ovum of the canine; and

(5) transplanting the fertilized ovum of the canine into the less bleeding side of fallopian tubes of a female canine of which both fallopian tubes have been embryo flushed.

The targeting site sequence can be determined directed to exon2 (Exon2: SEQ ID NO: 4), exon3 (Exon3: SEQ ID NO: 5) or exon4 (Exon4: SEQ ID NO: 6) of the APOE gene sequence. The targeting site sequence in the present example is the following sequence determined directed to exon3 (Exon3: SEQ ID NO: 5):

(SEQ ID NO: 7)
5′-CCGGGTGGCAGACTGGCCAGCCC-3′.

The sgRNA sequence and the complementary sequence synthesized in step (2) of the present example are:

sgRNA sequence:
(SEQ ID NO: 8)
ataGGGCTGGCCAGTCTGCCACCgt;
and
sgRNA complementary sequence:
(SEQ ID NO: 9)
taaaacGGTGGCAGACTGGCCA GCC.

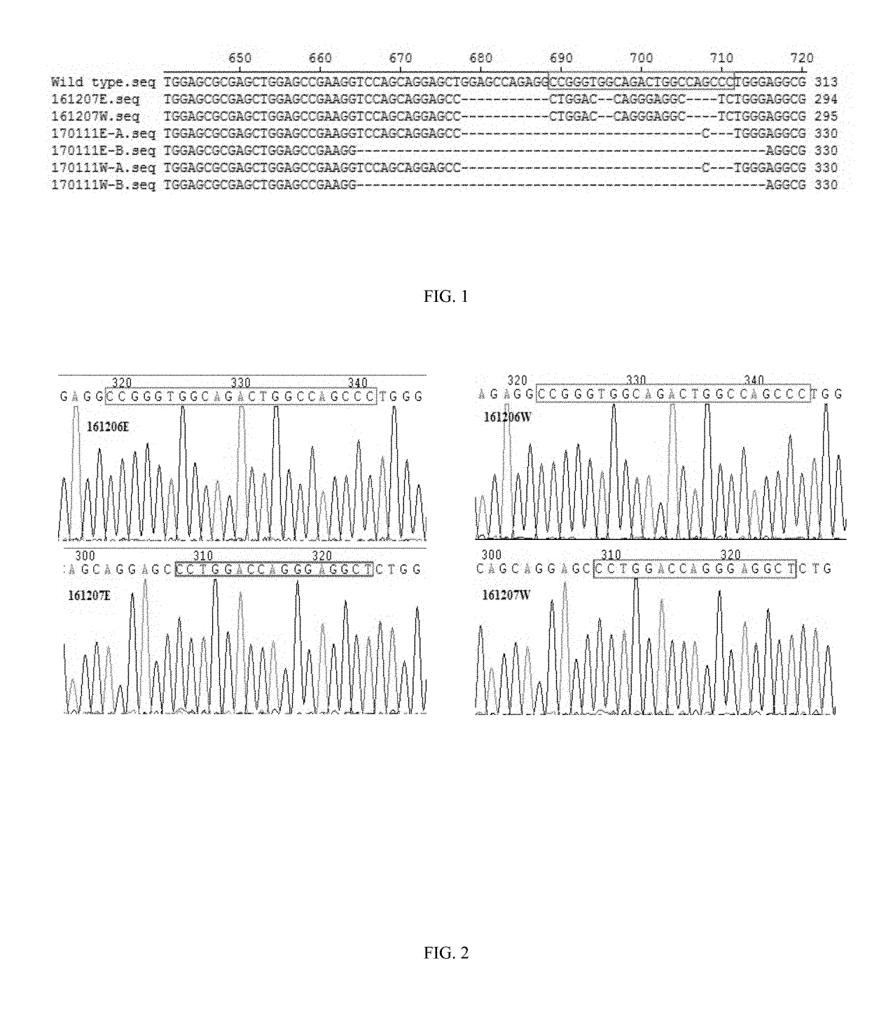

The embryo transplanting has been carried out totally 13 times, transplanted to 13 receptors, and totally 13 baby canines are born (see the following Table 1), wherein the APOE gene mutating type of the male canine named Apple (161207) is that a fragment of 34 bp was deleted and a fragment of 17 bp was inserted at the same time, the canine is APOE gene homozygous double knock-out canine, the APOE protein starts mutation from the 37^{th} amino acid, and the translation is terminated at the 63^{th} amino acid (please refer to FIG. 1). FIG. 1 shows a result of APOE gene knock-out from ear tissue and tail tissue of two gene knock-out canines numbered 161207 and 170111 respectively compared with the wild canine. It can be seen that the ear tissue and tail tissue of the gene knock-out canine named Apple numbered 161207 have the featured sequence:

(SEQ ID NO: 1)
cctggaccagggaggc.

FIG. 2 shows a comparison of gene sequence peaks of ear tissue and tail tissue of the APOE gene knock-out canine-Apple numbered 161207 to those of the wild canine numbered 161206. It is seen that ear tissue and tail tissue of the APOE gene knock-out canine-Apple numbered 161207 also possess the featured sequence:

TABLE 1
Result of Embryo Transplanting

| | Numbers of | Numbers | Numbers of Gene |
|---|---|---|---|
| Receptor | Fertilized Ovum | of | Knock-Out |
| Canine No. | as Transplanted | Offspring | Canine |
| FRA1115 | 8 | 0 | 0 |
| FRA1121 | 4 | 0 | 0 |
| FRA1126 | 5 | 0 | 0 |
| FRA1118 | 1 | 0 | 0 |
| FRA1124 | 5 | 4 | 1 |
| FRA1123 | 6 | 1 | 0 |
| FRA1129 | 7 | 1 | 0 |
| FRA1024 | 8 | 0 | 0 |
| FRA1130 | 6 | 2 | 0 |
| FRA1139 | 2 | 1 | 0 |
| FRA1137 | 2 | 6 | 1 |
| FRA1140 | 8 | 0 | 0 |
| FRA1146 | 3 | 1 | 0 |
| Total | 65 | 13 | 2 |

(SEQ ID NO: 1)
cctggaccagggaggct

Blood lipid detection of APOE gene knock-out canine:

At three month old, blood is collected from the APOE gene knock-out canine-Apple (161207), and is centrifugated for separating serum. The contents of total cholesterol, triglyceride, high-density lipoprotein and low-density lipoprotein in serum were measured. The results show that compared with control canines (numbered 161205 and 161206, respectively), the contents of total cholesterol, triglyceride, high density lipoprotein and low density lipoprotein in serum of APOE gene knock-out canine are apparently higher than those for the control group (Table 2). It can be seen that the knocking-out of APOE gene causes abnormal metabolism of lipids in the gene knock-out canine, leading to significant increase of blood lipid, which further verifies that the present invention indeed obtains the APOE gene knock-out canine.

TABLE 2
Blood lipid detection result of APOE gene knock-out canine

| Canine No. | 161207(APOE-/-) | 161205(WT) | 161206(WT) |
|---|---|---|---|
| Total cholesterol | 22.92 | 7.225 | 8.25 |
| (mmol/L) | | | |
| Triglyceride (mmol/L) | 2.25 | 1.505 | 0.86 |
| High-density lipoprotein | 8.80 | 5.535 | 6.08 |
| (mmol/L) | | | |
| Low density lipoprotein | 13.10 | 1.15 | 1.78 |
| (mmol/L) | | | |

(2) Preparing a Somatic Cell of APOE Gene Knock-Out Canine

The Beagle canine-Apple (161207) prepared in the above (1) is the first APOE gene knock-out canine in the world (the fibroblast BGD-APOEKO-EF0 having all the genetic information thereof is deposited in China General Microbiological Culture Collection Center (CGMCC) on Mar. 1, 2017 with a CGMCC deposit No. 13804) prepared by Beijing Sinogene Biotechnology Co. Ltd. When taking skin tissues, using a shaving knife to shave fur around ear edge tissue of the canine to be collected, sterilizing with ethyl alcohol of 75%, scissoring about 1 cm² of ear edge tissue of the canine with a sterilized scissor, putting thereof into a DMEM basic medium containing 100 IU/ml penicillin+100 IU/ml streptomycin, and taking it back to laboratory within 12 hrs.

The tissue block is first washed three times or more with PBS, and then washed 3 times with DMEM medium containing 5× double resistant (penicillin and streptomycin), fat tissue is removed carefully with an ophthalmic scissor for exposing dermal tissue. The skin tissue is transferred into another sterile culture container, and is cut with a scalpel into tissue blocks sized about 1 mm². The tissue blocks are transferred to the bottom of the culture container with a ophthalmic forceps, spread out evenly with the tip of the gun, and then the culture container is turned over with addition of DMEM culture liquid containing FBS of 20% and 5× double resistant, and cultured in an incubator with 37° C., 5% CO₂ and 100% humidity. After 7-8 hours, turning over the container, when the tissue blocks are adherent such that they are immersed completely by the medium for further culturing, the culture liquid is changed every 48 hours.

(3) Oestrous Identification of a Female Canine

In the present example, altogether 16 donor and receptor canines of oocytes are used, and oestrous identification is carried out through vagina smear and serum progesterone detection. Vagina smear is carried out every day for female canines entering the oestrous period, which includes inserting a saline infiltrated cotton rod of 5 cm long into vagina, smearing the sample onto a glass slide, after stained with Giemsa dye, and counting keratinization degree of the vagina epithelial cell under a microscope. When the keratinization degree is 80-90%, it is considered to be in the ovulatory period. When detecting the progesterone, 3 mL blood is collected and centrifugated for separating serum, a progesterone detecting instrument is used to detect progesterone content. When the progesterone value reaches 4-7 ng/mL, it is considered to be in the ovulatory period, after 72-120 hours of ovulation, matured oocytes are taken through flushing.

(4) Obtaining Matured Oocytes

First, 0.1 ml Quanmianbao (QFM mixture) is used to induce anesthesia of female canines, then isoflurane and ventilator are used to maintain anesthesia. The uterine tube conjunct area of ovary and uterus is exposed, a metal injecting needle having a round front end is inserted into the fallopian tube umbrella from the rupture of the ovarian bursa, and then the needle tube is fixed; an injecting needle is inserted into a fallopian tube at the uterine tube conjunct area, flushing the fallopian tube with TCM199 medium containing 10% FBS, a plastic tube of 3 cm long is punctured inside the uterus, fixed, and then flushing the ovum with embryo flushing liquid from the uterine tube conjunct area. The embryo flushing can be carried out at a reversed direction, but the needle tube at the end for receiving the embryo flushing liquid should have a diameter greater than that of the needle tube for inputting embryo flushing liquid. Female canines selected as receptors are subjected to embryo flushing at only one side of the fallopian tubes, the other side is reserved for embryo transplanting. Female canines are for reviving treatment at once when the embryo flushing is terminated, and is anesthesia again at the time of embryo transplanting. Other donor canines that only provide oocyte are subject to embryo flushing at both sides of the fallopian tubes, the obtained oocyte are used for somatic cell nucleus transplanting.

(5) Oocyte Ennucleation

The in vovo or in vitro collected matured oocyte is put into a DPBS solution containing 0.1% hyaluronidase, blowing and beating repeatedly with a pipette on a hot stage of 37° C. to remove cumulus cell. To observe under an inverted microscope by amplifying 200 times to select matured oocyte containing the first polar body.

The selected oocyte is put into HEPES buffered CR2aa microoperating liquid containing 10% FBS and 5 μg/ml cytochalasin B, incubated 30 min to soften the cytoskeleton, then the first polar body, adjacent cytoplasm (less than 5%) and oocyte nucleus are removed with a microscopy injecting needle, then the enucleated oocyte is reserved in SOF medium. The first polar body and cytoplasm sucked out by the microscopy injecting needle are put into Hochest 33342 solution for staining, then observing whether the sucked cytoplasm contains cell nucleus under a fluorescent microscope and judging the enucleating efficiency. If the enucleating efficiency is above 90%, somatic cell injection can be carried out, otherwise, oocyte is stained with Hochest 33342, UV irradiated under fluorescent microscope to clearly remove the indicated cell nucleus.

(6) Nuclear Injection, Fusion and Activation of the Enucleated Oocyte

The enucleated oocyte is placed in a CR2aa microoperating liquid without CB, and then the selected donor cell is injected between zona pellucid and cytoplasm of the oocyte, the zona pellucid is point pressed slightly by the injecting needle such that the somatic cell and oocyte membrane bond closely for constructing reconstructed embryo. The reconstructed embryo is put into a fusion liquid (containing 0.24M mannitol, 0.1 mM MgSO4, 0.5 mM Hepes and 0.05% BSA) having an osmotic pressure of 240 mOSM, then is placed between paralleled electrodes of the fusion groove for electrofusion using ECM2001 (BTX) under a voltage of 3-3.6 kv/cm, pulsing two times, and under a condition of a pulsing interval of 10 μs. After 30 min of fusion, using a stereomicroscope to observe the fusion of cytoplasm of oocyte and donor cell, and count the fusion efficiency. The reconstructed embryo identified as fusion is activated with a 10 μmol/L ionomycin for 4 min, further activated with mSOF containing 2 mmol/L 6-DMAP for 4 hours, after the activating, embryo transplanting is under preparation. See the following Table 3, it can be seen that during embryo fusion, using a fusion liquid having an osmotic pressure of 24 mOsm can maintain a fusion ratio of above 70%; while the fusion ratio is only about 50% with a commonly used fusion liquid having an osmotic pressure (300 mOsm) in the prior art.

TABLE 3
Influence of fusion liquid osmotic pressure on fusion ratio

| Fusion Liquid Osmotic | Times of Electro | Number of | Fusion |
|---|---|---|---|
| Pressure (mOsm) | shock to embryo | Fusion Embryo | ratio |
| 240 | 61 | 45 | 73% |
| 300 | 43 | 7 | 53.4% |

(7) Embryo Transplanting

After anaesthesia in the receptor canines, the abdominal wall that was not embryo flushed is selected and the ovary tissue that was not embryo flushed is exposed, and the uterus and ovary are pulled out. The cloned embryo is sucked into an embryo transplanting tube; the embryo transplanting tube is inserted from umbrella portion of the fallopian tube for transplanting the embryo into the receptor. Referring to Table 4, B ultrasonic examination is carried out after 25 days of embryo transplanting, the embryo transplanting result shows that using autologous/allogenic embryo transplanting significantly increases pregnant rate. The present experiment uses totally 4 transplanting receptors, of which 2 are pregnant and altogether 3 cloned canines are born.

TABLE 4
Summary of embryo transplanting

| | No. of | | No. of the | | |
|---|---|---|---|---|---|
| | Donor | | Born | | |
| No. of | Canines | No. of | Cloned | Number and | |
| Receptor | (Including | Transplanted | Baby | Name of the Cloned | |
| Canine | receptors) | Embryo | Canines | Baby Canines | |
| NTR1217 | 9 | 21 | 1 | 170502(Long Long) | |
| NTR1243 | 2 | 7 | 2 | 170610 | 170611 |

| | | | | |
|---|---|---|---|---|
| NTR1252 | 2 | 9 | 0 | — |
| NTR1256 | 3 | 7 | 0 | — |
| In Total | 16 | 44 | 3 | — |

(8) Microsatellite Identification of the Cloned Canines

In order to determine whether the born cloned baby canines and the somatic cell donor canine-“Apple” (numbered: 161207) have the same genetic information, and determine the genetic relationship with the embryo transplanting receptor female canine, the method of microsatellite identification is used to identify the genetic relationship of the above three. 14 microsatellite sites are selected, and are: PEZ2, PEZ3, PEZ5, PEZ6, PEZ8, PEZ12, PEZ15, PEZ20, PEZ21, FH2011, FH2054, FH2079, FH2132, FH2611 and VWFX. This identification was implemented by DNA laboratory of Nanchang Patrol Dog Base of Ministry of Public Security.

In the first identification, the submitted sample from Apple numbered 161207 is used as the sample No. 1, the cloned baby canine Long Long numbered 170502 delivered by the receptor canine NTR1217 is used as the sample No. 2, and the receptor canine NTR1217 is used as the sample No. 3; the identity between sample No. 2 and sample No. 1 is identified, and the parenthood between the sample No. 2 and the sample No. 3 is identified.

Using a sampler to take two parts from each sample as parallel controls, and a canine STR fluorescent detecting kit is used for PCR amplification. The amplification product is subjected to electrophoresis and typing with use of AB131030 genetic analyzer.

STR polymorphism testing result: upon comparison, the STR typing of canine sample No. 2 and the canine sample No. 1 is consistent; and the canine sample No. 2 and the canine sample No. 3 do not match at multi-locus including PEZ2, FH2054, FH213 and PEZ15.

In the second identification, the submitted sample of Apple numbered 161207 is used as the sample No. 2, the cloned baby canine numbered 170610 delivered by the receptor canine NTR1243 is used as the sample No. 3, the cloned baby canine numbered 170611 delivered by the receptor canine NTR1243 is used as the sample No. 4, and the receptor canine NTR1243 is used as the sample No. 1; identifying the identity of the sample No. 1, the sample No. 3, the sample No. 4 and the sample No. 2, and the parenthood between the sample No. 3 and the sample No. 1.

Using a sampler to take two parts from each sample as parallel controls, and a canine STR fluorescent detecting kit is used for PCR amplification. The amplification product is subjected to electrophoresis and typing with use of AB131030 genetic analyzer.

STR polymorphism testing result: upon comparison, the locus data of samples No. 3 and No. 4, and the sample No. 2 are matched; and samples No. 3 and No. 4, and the sample No. 1 do not match at multi-locus including PEZ12, PEZ15, etc.

The identification result shows that the cloned canine “Long Long” numbered 170502, the cloned canine numbered 170610, the cloned canine numbered 170611 and the cell donor “Apple” numbered 161207 have consistent typing at all microsatellite sites, but are not matched with the receptor canines NTR1217 and NTR1243 for embryo transplanting at sides including PEZ2, PEZ5, PEZ6, PEZ8, PEZ12, PEZ15, PEZ20, FH2011, FH2054, FH2079, FH2132, FH2611 and VWFX (referring to the following Table 5), proving that the cloned canine “Long Long” numbered 170502, the cloned canine numbered 170610 and the cloned canine numbered 170611 are the cloned canines of the cell donor canine “Apple” numbered 161207. FIG. 4 is a comparison picture of the APOE gene knock-out canine-Apple (FIG. 4A) numbered 161207 to the cloned canine “Long Long” (FIG. 4B) numbered 170502, 30 days after birth.

TABLE 5
the microsatellite identification result of the cloned canines

| | Canine No. | | | | | |
|---|---|---|---|---|---|---|
| | | | | | NTR1217 | NTR1243 |
| | 161207 | 170502 | 170610 | 170611 | (embryo | (embryo |
| Microsatelite | (Apple, | (Long Long, | (cloned | (cloned | transplanting | transplanted |
| site | donor canine) | cloned canine) | canine) | canine) | receptor) | receptor) |
| PEZ2 | 171/171 | 171/171 | 171/171 | 171/171 | 183/183 | 159/183 |
| PEZ3 | 185/185 | 185/185 | 185/185 | 185/185 | 185/185 | 185/185 |
| PEZ5 | 262/266 | 262/266 | 262/266 | 262/266 | 262/262 | 262/262 |
| PEZ6 | 263/263 | 263/263 | 263/263 | 263/263 | — | 263/267 |
| PEZ8 | 344/349 | 344/349 | 344/349 | 344/349 | 340/349 | 344/351 |
| PEZ12 | 296/308 | 296/308 | 296/308 | 296/308 | 296/308 | 298/308 |
| PEZ15 | 178/186 | 178/186 | 178/186 | 178/186 | 182/192 | 193/193 |
| PEZ20 | 120/124 | 120/124 | 120/124 | 120/124 | 120/120 | 120/128 |
| PEZ21 | 132/132 | 132/132 | 132/132 | 132/132 | 132/132 | 132/132 |
| FH2011 | 215/215 | 215/215 | 215/215 | 215/215 | 215/223 | 215/215 |
| FH2054 | 130/130 | 130/130 | 130/130 | 130/130 | 126/138 | 122/126 |
| FH2079 | 263/263 | 263/263 | 263/263 | 263/263 | 275/275 | 275/275 |
| FH2132 | 282/298 | 282/298 | 282/298 | 282/298 | 290/330 | 282/300 |
| FH2611 | 236/244 | 236/244 | 236/244 | 236/244 | 236/238 | 236/236 |
| VWFX | 186/192 | 186/192 | 186/192 | 186/192 | 192/192 | 186/192 |

(9) APOE Gene Knock-Out Identification of the Cloned Canines

In order to determine whether the cloned baby canines are APOE gene knock-out canines, i.e., the same as the somatic cell donor canine-“Apple” (numbered 161207). The tail tissues of the cloned canine “Long Long” numbered 170502, and the cloned canine numbered 170610 and 170611 respectively are used for identification. After the tissue block is fragmented in a centrifugation tube, protease K is added for water bath at 56° C. and cleavage for 1-3 h. Then 700 μl of Genomic Lysis Buffer sucked with a pipette is added to the cleavage system, mixing homogeneously through turning upside down, and then 10,000 g centrifugation for 1 min. The supernatant was sucked to a purifying column with a pipette, 10,000 g, standing for 1 min at room temperature. A new collection tube was replaced, and 200 μl DNA Pre-Wash Buffer was added to the centrifugation column, 10,000 g, followed by standing for 1 min at room temperature, centrifuging for 1 min, and discarding wasted liquid. 400 μl g-DNA Wash Buffer was added to the centrifugation column, 10,000 g, standing for 1 min at room temperature, centrifuging for 1 min, and discarding the wasted liquid. The purifying column and the collecting tube are re-centrifugated, 10,000 g, centrifuging for 2 min. The purifying column was placed in a newly replaced 1.5 ml centrifugation tube, 50 μl Elution Buffer was added to elute DNA, followed by standing for 2 min at room temperature, and then 12,000 rpm, centrifuging for 1 min. The obtained solution is canine genomic DNA.

The canine genomic DNA was used as a template to carry out PCR, and the primers are as follows:

(SEQ ID NO: 10)
F: 5′-CATTGTTGTCAGGCAGGTAGC-3′;
(SEQ ID NO: 11)
R: 5′-GAAGGGTGCGAGGGATTGA-3′.

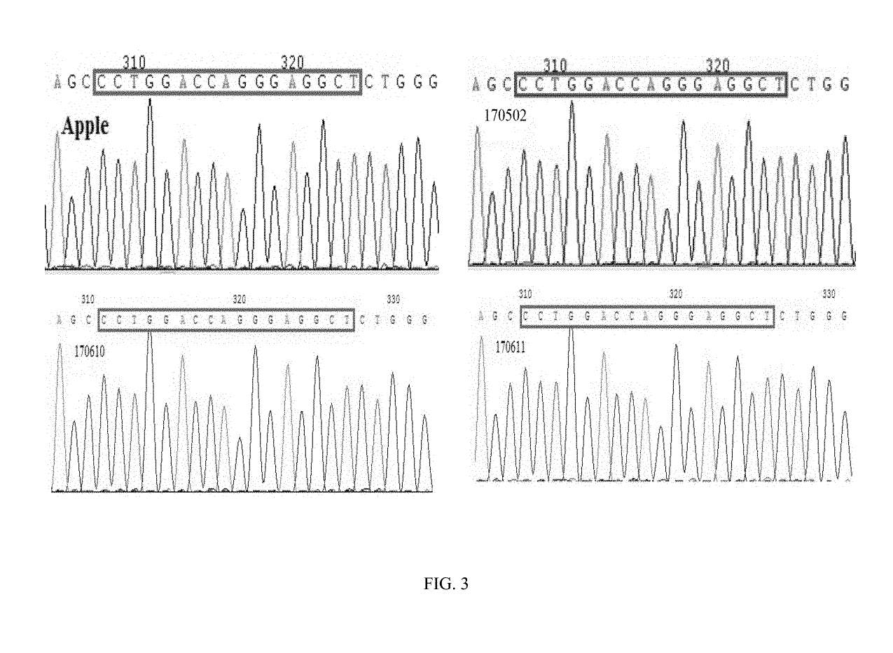

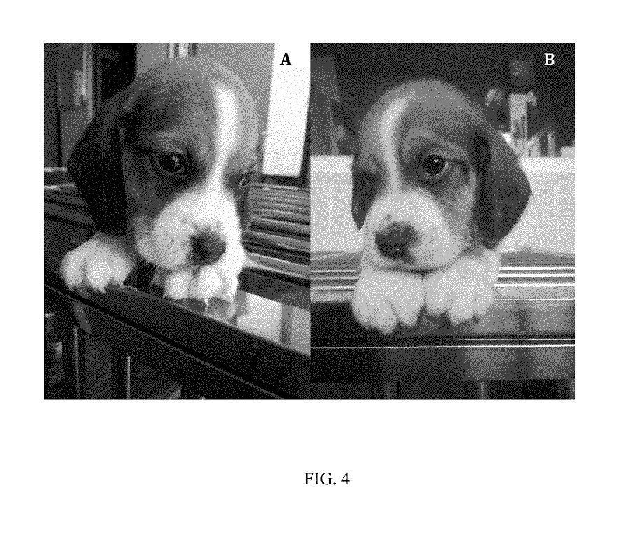

After amplification, a DNA fragment of total 660 bp at the upstream and downstream of the recognition and cleavage targeting site of sgRNA. The target fragment obtained by PCR amplification was subjected to DNA sequencing, and aligned with the canine APOE gene sequence provided by the NCBI database to determine the mutation type of the APOE gene. The result shows that the cloned canine “Long Long” numbered 170502 and the cloned canines numbered 170610 and 170611 respectively have consistent APOE gene mutating type with the somatic cell donor canine named Apple and numbered 161207 (referring to Table 6 and FIG. 3 and FIG. 4).

TABLE 6
Alignment of the gene mutating sequences for the cloned canines

| | | Gene Mutating |
|---|---|---|
| Gene Type | Targeting Sequence | Type |
| wild type | AGCTGGAGCCAGAGGCCGGGTGGCAGACTGGCCAGCCCTG | |
| | GGAGGCGGCGCTG | |
| 161207(Apple) | AGC-------------------- | Deleting 34 bp, |
| | CCTGGACCAGGGAGGCT----------CTGGGAGGCGGCGCTG | Adding 17 bp |
| 17050(Long | AGC-------------------- | Deleting 34 bp, |
| Long) | CCTGGACCAGGGAGGCT----------CTGGGAGGCGGCGCTG | Adding 17 bp |
| 170610 | AGC-------------------- | Deleting 34 bp, |
| | CCTGGACCAGGGAGGCT----------CTGGGAGGCGGCGCTG | Adding 17 bp |
| 170611 | AGC-------------------- | Deleting 34 bp, |
| | CCTGGACCAGGGAGGCT----------CTGGGAGGCGGCGCTG | Adding 17 bp |

It can be seen from the above result that preparing gene knock-out canines with somatic cell nucleus transplanting technology and using somatic cell identified to be gene knock-out as nuclear donor, all the offspring cloning canines are gene knock-out, having a knock-out efficiency of up to 100%. The mutating type can be determined by identifying the donor cell, and the gene knock-out effect is stable without existence of any chimera by selecting the somatic cell, whose targeting gene is completely silenced, for cloning.

What is claimed is:

1. A somatic cell cloning method of a gene knock-out canine, comprising the following steps:
(1) preparing a targeting gene knock-out somatic cell as a nuclear donor;
(2) preparing an enucleated oocyte from a receptor female canine;
(3) introducing a gene knock-out somatic cell into cytoplasm of the enucleated oocyte and constructing a cloned embryo;
(4) activating the cloned embryo;
(5) transplanting the cloned embryo obtained in step (4) to the receptor female canine;
wherein the enucleated oocyte is obtained from the embryo flushed fallopian tube of a receptor female canine of which only one of fallopian tubes has been embryo flushed, and, in step (5), the cloned embryo is transplanted into the fallopian tube of the receptor female canine, which is not embryo flushed.

2. The method according to claim 1, wherein in step (2) for preparing the enucleated oocyte from the receptor female canine, a female canine having a keratinocyte ratio above 80-90% and a progesterone level up to about 4-7 ng/mL is used as a receptor female canine under ovulatory period.

3. The method according to claim 2, wherein after 72 h-120 h of ovulation, single side embryo flushing is carried out to obtain a matured oocyte, then enucleating the matured oocyte.

4. The method according to claim 1, wherein the targeting gene knock-out somatic cell is a somatic cell of a gene knock-out canine obtained by knocking-out an endogenous gene of a canine oosperm with the use of gene knock-out technology, and the somatic cell is a gene knock-out somatic cell whose targeting gene is completely silenced via identification.

5. The method according to claim 1, wherein the targeting gene knock-out somatic cell is a gene knock-out somatic cell obtained by knocking-out an endogenous gene of a canine somatic cell with the use of gene knock-out technology, and selecting the gene knock-out somatic cell, whose targeting gene is completely silenced via identification.

6. The method according to claim 1, wherein the gene knock-out technology of the targeting gene knock-out is selected from clustered regularly interspaced short palindromic repeat sequences technology (CRISPR/Cas9), Zinc finger nuclease technology (ZFN), transcriptional activator effector nuclease technology (TALENs) and homologous recombination technology.

7. The method according to claim 1, wherein in step (3), the gene knock-out somatic cell is introduced into the cytoplasm of the enucleated oocyte through electrofusion with use of a fusion liquid having an osmotic pressure of 200 mOSM-280 mOSM.

8. The method according to claim 7, wherein in step (3), the gene knock-out somatic cell is introduced into the cytoplasm of the enucleated oocyte through electrofusion with use of a fusion liquid having an osmotic pressure of 240 mOSM.

9. The method according to claim 7, wherein the fusion liquid is composed of 0.2-0.28M mannitol, 0.1 mM MgSO4, 0.5 mM Hepes and 0.05% BSA.

10. The method according to claim 1, wherein in step (3), the gene knock-out somatic cell is introduced into the cytoplasm of the enucleated oocyte through electrofusion under a voltage of 2-4 kv/cm.

11. The method according to claim 1, wherein the somatic cell is from the following tissues or organs: fetal tissue, skin, muscle, ear, breast, fallopian tube, ovary, blood, urine, fat, marrow, blood vessel and endothelium of the lumen.

12. The method according to claim 11, wherein the somatic cell is selected from fetal fibroblast, skin cell, epithelial cell, ear cell, fibroblast, endothelial cell, muscle cell, breast cell, fallopian tube cell, ovary cell, cumulus cell, nerve cell and osteoblast.

13. The method according to claim 1, wherein the method comprises the following steps:
(1) preparing an APOE gene knock-out somatic cell as a nuclear donor;
(2) preparing an enucleated oocyte from a receptor female canine;
(3) introducing a gene knock-out somatic cell into the cytoplasm of the enucleated oocyte and constructing a cloned embryo;
(4) activating the cloned embryo;
(5) transplanting the cloned embryo obtained in step (4) into the receptor female canine;
wherein APOE gene Exon3 of the APOE gene knock-out somatic cell comprises the following sequence:
(SEQ ID NO: 1)
cctggaccagggaggct;
the enucleated oocyte is obtained from the embryo flushed fallopian tube of a receptor female canine of which only one of fallopian tubes has been embryo flushed, and, in step (5), the cloned embryo is transplanted into the fallopian tube of the receptor female canine, which is not embryo flushed.

14. The method according to claim 13, wherein the APOE gene Exon3 of the APOE gene knock-out somatic cell comprises the following sequence:
(SEQ ID NO: 2)
ctggagcgcgagctggagccgaaggtccagcaggagccctggaccaggga
ggctctgggaggc.

15. The method according to claim 13, wherein the APOE gene Exon3 of the APOE gene knock-out somatic cell comprises the following sequence:
(SEQ ID NO: 3)
gatgctgggccgatgtgcagccggagccggagctggagcgcgagctggag
ccgaaggtccagcaggagccctggaccagggaggctctgggaggeggcgc
tggcccgcttctgggattacctgcgctgggtgcagacgctgtctgaccag
gtgcaagagggcgtgctcaacacccaggtcacccaggaactgac.

16. The method according to claim 13, wherein the APOE gene knock-out somatic cell is ear fibroblast BGD-APOEKO-EF0 of the APOE gene knock-out beagle canine, which is deposited in China General Microbiological Culture Collection Center (CGMCC) on Mar. 1, 2017 with a CGMCC deposit No. 13804.


SEQUENCE LISTING (11 sequences)

SEQ ID NO: 1 - DNA, 17 bp, Canis lupus
cctggaccagggaggct

SEQ ID NO: 2 - DNA, 63 bp, Canis lupus
ctggagcgcgagctggagccgaaggtccagcaggagccctggaccagggaggctctgggaggc

SEQ ID NO: 3 - DNA, 194 bp, Canis lupus
gatgctgggccgatgtgcagccggagccggagctggagcgcgagctggagccgaaggtccagcaggagccctggaccagggaggctctgggaggcggcgctggcccgcttctgggattacctgcgctgggtgcagacgctgtctgaccaggtgcaagagggcgtgctcaacacccaggtcacccaggaactgac

SEQ ID NO: 4 - DNA, 43 bp, Canis lupus
atgaaggttctgtgggctgcgctggtggtcacgctcctggcag

SEQ ID NO: 5 - DNA, 211 bp, Canis lupus
gatgctgggccgatgtgcagccggagccggagctggagcgcgagctggagccgaaggtccagcaggagctggagccagaggccgggtggcagactggccagccctgggaggcggcgctggcccgcttctgggattacctgcgctgggtgcagacgctgtctgaccaggtgcaagagggcgtgctcaacacccaggtcacccaggaactgac

SEQ ID NO: 6 - DNA, 718 bp, Canis lupus
ggcgctgatggatgagaccatgaaggaggtgaaggcctacaaggcggagctggacgagcagctgggccccatgacctcggagacgcaggcccgcgtggccaaggagctgcaggcggcgcaggcccggctgcgctcggacatggaggacgtgcgcaaccgcctgacgcagtaccgcggcgagctgcaggccatgctgggccagagcagcgaggagctgcgggcgcgcttcgcctcccacatgcgcaagctgcgcaagcgggtgctgcgggacgccgaggacctgcagaggcgcctggccgtctacaaggccggcgtgcgcgagggtgccgagcgcagcgtgagcagcatccgcgagcgcctctggccgctgctggagcaggcccgcgagcgcaacgccaaggtgggcgccctggccacgcagccgctgctcgagcgggccgacgccctgggccagcagctgcgcgggcagctggaggagatgagcagccgggcccgcggccacctggaggagatgcgcgagcagatacaggaggtgcgggtgaagatggaggagcaggccgaccagatacgccaaaaggccgaggccttccaggcgcgcctcaagagctggttcgagcccctgctggaagacatgcagcgccagtgggacgggctggtggagaaggtgcaggcggccgtggccaccatccccacctctaagcctgtggaggaaccatga

SEQ ID NO: 7 - DNA, 23 bp, Canis lupus
ccgggtggcagactggccagccc

SEQ ID NO: 8 - DNA, 25 bp, Artificial Sequence
  sgRNA
atagggctggccagtctgccaccgt

SEQ ID NO: 9 - DNA, 25 bp, Artificial Sequence
  sequence complementary to sgRNA
taaaacggtggcagactggccagcc

SEQ ID NO: 10 - DNA, 21 bp, Artificial Sequence
  forward primer
cattgttgtcaggcaggtagc

SEQ ID NO: 11 - DNA, 19 bp, Artificial Sequence
  reverse primer
gaagggtgcgagggattga